(19) United States
(12) Patent Application Publication
(10) Pub. No.: US 2002/0015826 A1
(43) Pub. Date: Feb. 7, 2002

(54) Zwitterionic iodonium compounds and methods of application

(76) Inventors: Darryl Desmarteau, Clemson, SC (US); Vitorio Montanari, Milano (IT); Brian H. Thomas, Central, SC (US)

Correspondence Address: John E. Vick, Jr., Dority & Manning, Attorneys at Law, P.A., P.O. Box 1449, Greenville, SC, 29602, US

(21) Appl. No.: 09/832,174

(22) Filed: Apr. 10, 2001

Related U.S. Application Data
(60) Provisional application No. 60/196,515, filed on Apr. 11, 2000.

(51) Int. Cl. C07C 25/02; B32B 3/30; C07C 381/00; C07C 321/00; C07C 17/00; C07C 323/00; C07C 319/00; C07C 323/09

(52) U.S. Cl. 428/195; 564/305; 568/56; 570/181

(57) ABSTRACT

Photoresist compositions, or photoacids, are used in etching circuit pathways on the surface of microelectronic components. Iodonium compounds generate a strong acid under short wavelength irradiation, which is capable of etching the polymeric surface coating of a silicon wafer. The strong acid is the conjugate acid of the counteranion. Larger counterions generate acids that diffuse to a lesser extent in the polymer resist matrix, producing better image resolution. Given the particular anion, the solubility of its diary iodonium salts will depend upon which aromatic rings are used on the iodonium molecule. A zwitterionic structure of the iodonium molecule, having both positive and negative charges on the same molecular species, can be used as photoacid composition in the photoresist step of microchip manufacturing.

### REFERENCE TO PREVIOUS APPLICATION

This application claims priority from previously filed Provisional Application No. 60/196,515 filed on Apr. 11, 2000.

### FIELD OF THE INVENTION

This invention is directed to compounds and processes that are useful in etching of microelectronic components. In particular, the invention relates to the generation of photoacids used in the photoresist step of microelectronic manufacture.

### BACKGROUND OF THE INVENTION

In the manufacture of electronic semiconductor devices, it is common to use silicon wafers as a base material. Such wafers may be etched to produce an integrated circuit. A modern integrated circuit is a three dimensional structure of alternating patterned layers of conductors, dielectrics, and semiconductor films. These circuits are usually fabricated upon an ultra-high purity wafer substrate of a semiconducting material such as silicon.

In many applications, silicon wafers are manufactured in a two step process. The first step is the coating of the silicon wafer with a thin film of a radiation sensitive polymer. This polymer is sometimes known as a “resist”. A second step is the transfer of a predetermined pattern to the silicon wafer substrate using an etching technique.

Conventional etching techniques sometimes have relied upon diazonaphthoquinone and novolac resins to perform the etching. Such resins have been successful in generating circuits having features or circuit path widths greater than about 0.8 μm. U.S. Pat. No. 5,945,250 to Aoai et al. describes positive photosensitive compositions containing a sulfonium or iodonium salt resin having a specific repeating structural unit with good stability in solvents and high photosensitivity for etching a resist pattern. The Aoai patent disclosure references novolac resins and napthoquinonediazide compounds that function as dissolution inhibiting agents. According to the Aoai patent disclosure, such photoresists produce satisfactory results in forming resist patterns having line widths ranging from about 0.8 to about 2 μm.

In general, resins operate upon the silicon wafer by undergoing a photochemical transformation that renders them soluble in aqueous base. Thus, the irradiated area on the surface of the wafer may be removed by exposing a pattern on the silicon underneath. Such etching systems sometimes have employed electromagnetic radiation having a wavelength of about 350-450 nm. Lithography which uses this wavelength of electromagnetic radiation in the etching process is sometimes called “conventional lithography”. This process uses photoacids, which are molecules that produce an acid when they are irradiated. Photoacids are used in the photoresist step of microchip manufacturing.

Recently, there has been a drive in the industry towards smaller components. In general, the smaller the overall electronic device, the greater the performance. In order to increase performance, it is necessary to place a more complex circuit on a wafer in a smaller space. To do so, it is necessary to manufacture a more exact and highly refined resolution of the photo-etch upon the surface of the wafer. To increase the resolution of the etch upon the wafer, it is necessary to employ a chemical etch system that is capable of operating at lower wavelengths of electromagnetic radiation.

In general, the shorter the wavelength used to produce the image, the higher the resolution that may be achieved on the surface. Therefore, to obtain a lithography etch with a circuit path width of less than about 0.5 μm, improved lithographic methods and compositions need to be developed that are capable of employing shorter wavelengths of electromagnetic radiation in the etching process.

Photoresist systems in the prior art are divided generally into two groups: two-component and three-component systems. The two-component systems contain a resin, which decomposes upon irradiation with light to give an alkaline soluble component, and a photoacid generator. The three component systems involve a photoacid generator, an alkali-soluble resin with acid-decomposable groups, and a dissolution inhibitive compound for the alkali-soluble resin. After irradiation, the acid-catalyzed removal of acid decomposable groups is carried out by heating; it is known that for each molecule of acid generated, many acid decomposable groups are removed during the heating stage. A discussion of this system is set forth in U.S. Pat. No. 5,945,250 to Aoai.

Recent work on photoacid generators has sometimes focused upon diaryl iodonium salts of the general formula (Ar)₂I⁺X⁻ and the sulfonium salts of perfluorinated Lewis acids. In particular, hexafluoroantimonates and hexafluoroarsenates have found significant use as photoacid generators. However, metals or heavy elements such as phosphorus, arsenic, and antimony are problematic, as they may pollute the semiconductor materials during the lithography process.

Some prior compositions and methods have focused upon sulfonium or iodonium compounds that employ counter anions such as trifluoromethanesulfonate and toluenesulfonate. When irradiated, these compounds form their corresponding sulfonic acids. However, some sulfonic acids diffuse too rapidly through the resist film on the surface of the substrate, and the resolution of the resist pattern produced from these rapidly diffusing photoacid generators is rendered unacceptable. Silicon wafers prepared using such acid generating systems suffer from a significant line width increase over time, and from other surface problems upon heat treatment.

Larger counter anions, such as the toluenesulfonates, may have relatively lower diffusion rates. However, such counter anions often have low solubility in resist solvents and thus do not provide a desirable resist pattern. It is therefore apparent that a photoacid generator in a photoresist system that does not allow the acid generated to undesirably migrate or diffuse through the polymer at unacceptably high rates would be advantageous.

Some resins are unsuitable for use in this manner because the compounds absorb UV radiation which is directed upon them, causing the dissipation of the UV radiation before it reaches the lower side of the layer. Conventional novolac resins and naphthoquinonediazide compounds are generally unsuitable for lithography that uses shorter wavelength sources (i.e. “deep UV”) because such compounds exhibit intense absorption in the far ultraviolet region. That is, UV light impacting the surface of the resist is not capable of reaching and striking the lower or deeper regions of the resist. As a result, the pattern of the novolac resin system which results when using far ultraviolet light with such compounds is sometimes undesirably tapered on the surface of the wafer, due to incomplete photoactivation throughout the wafer.

Thus, a resist system employing a photoacid that is capable of providing for a high quality lithography etch having a fine resolution of less than about 0.5 μm on the wafer surface, facilitating the use of shorter wavelengths of electromagnetic radiation in the etching process, would be highly desirable. An etching system that provides a good etch, but does not show a high degree of undesirable absorbance in the low UV portion of the spectrum, is attractive and desirable for producing a precise etch on a substrate.

### BRIEF DESCRIPTION OF THE DRAWINGS

A full and enabling disclosure of this invention, including the best mode shown to one of ordinary skill in the art, is set forth in this specification. The following Figures illustrate the invention:

FIG. 1 shows a typical prior art method of synthesizing an aryl iodonium compound;

FIG. 2A is a zwitterionic compound in one embodiment of this invention which includes two aryl groups attached to a positively charged iodine atom, and a negatively charged group on one of the aryl atoms;

FIG. 2B depicts another compound (i.e. embodiment) of this invention;

FIG. 2C shows a compound of the invention substituted with a —CF₃ group;

FIG. 3A shows a generic structure for an intermediate used to synthesize the zwitterionic compounds of this invention;

FIG. 3B shows another chemical intermediate used to synthesize the zwitterionic compounds of this invention;

FIG. 3C shows yet another chemical intermediate used to synthesize the zwitterionic compounds of this invention;

FIG. 4A is one pathway for synthesizing compounds that are useful in the application of this invention;

FIG. 4B shows an alternate pathway for synthesizing compounds of this invention;

FIG. 5 shows a reaction used to combine two aryl groups;

FIG. 6A is a graphical representation of Thermal Gravimetric Analyses (TGA) of a typical iodonium zwitterion;

FIG. 6B is a graphical representation of TGA analysis of an iodonium zwitterion with DMSO of crystallization;

FIG. 7 shows a synthesis pathway for iodobenzenesulfonimide; and

FIG. 8 shows reactions used to produce several compounds of this invention.

### DETAILED DESCRIPTION OF THE INVENTION

Reference now will be made to the embodiments of the invention, one or more examples of which are set forth below. Each example is provided by way of explanation of the invention, not as a limitation of the invention. In fact, it will be apparent to those skilled in the art that various modifications and variations can be made in this invention without departing from the scope or spirit of the invention. For instance, features illustrated or described as part of one embodiment can be used on another embodiment to yield a still further embodiment. Thus, it is intended that the present invention cover such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention, which broader aspects are embodied in the exemplary constructions.

A photoresist system usually is comprised of a polymer coating applied on the substrate to be etched and a mixture of PAG and the photoresist formulation. The PAG then decomposes to generate an acid only in the areas irradiated. In general, an electronic device such as an integrated circuit is manufactured in a two step process. The first step is the coating of a silicon wafer with a thin film of radiation sensitive polymer (resist). The second step is the transfer of the pattern to the substrate using an etching technique. Conventional techniques rely upon diazonaphthoquinone and novolac resins, which have been reported to work for generating circuits with features greater than about 0.8 μm.

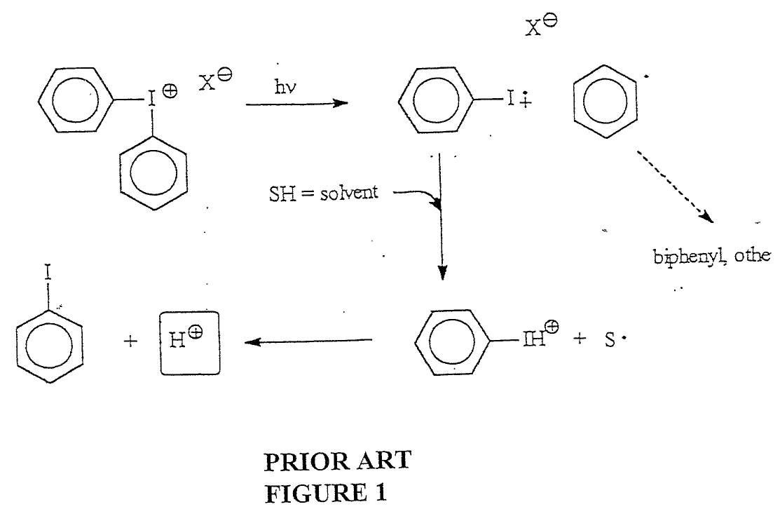

FIG. 1 shows a prior art conventional iodonium salt employed as photoacid generator. Two aromatic groups are attached to an iodine atom. There is a positive charge on the iodine atom, and a negative charge on the separate X⁻ group. In solvent, the salt separates into ions as shown in the lower portion of FIG. 1.

This invention, however, is directed to resist compositions that are capable of transmitting UV light of low or reduced wavelength. Iodonium salts generate a strong acid under short wavelength irradiation. The strong acid is the conjugate acid of the counteranion. Larger counterions generate acids that diffuse to a lesser extent in the polymer matrix (known as the “resist”), producing better image resolution. Given the particular anion, the solubility of its diaryl iodonium salts will depend on which aromatic rings are used.

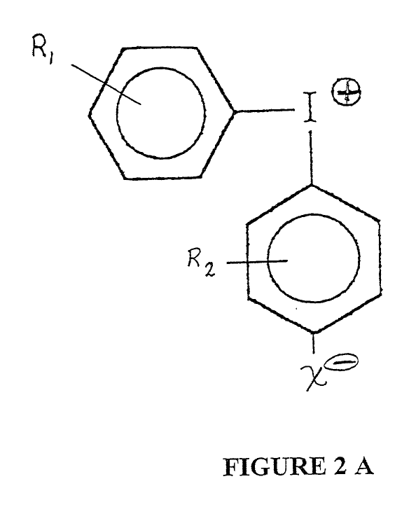

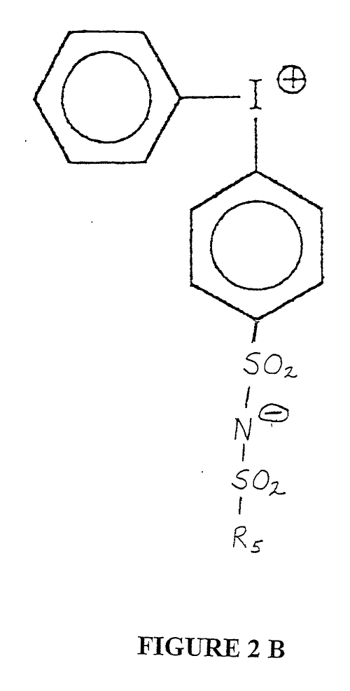

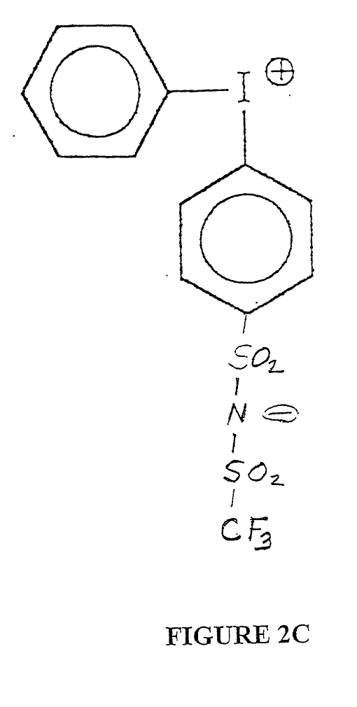

In one aspect of this invention, a compound having the zwitterionic structure shown in FIG. 2A is employed as a photoresist composition. This structure is a zwitterionic molecule, with both a positive and negative charge on the same species. There are two aromatic rings connected to an iodine atom, with a positive charge on the iodine and a negative charge on one of the moities attached to one of the aromatic groups (X⁻). Further, R₁ and R₂ represent groups that may be attached to the aromatic rings at any remaining carbon position on the respective rings. R₁ and R₂ are each independently selected from the group consisting of substituted or unsubstituted: alkyls, aryls, halides, and fluorinated alkyls, nitrogen containing groups, halogenated alkyls, alkoxy, aryloxy, halogenated alkoxy, unsaturated alkyls, thioalkyls, unsaturated fluorinated alkyls, unsaturated alkoxy keto alkyls, alkoxys, aryloxy, keto aryls, sulfonyl alkyl, sulfonyl aryls. Alternately, they may be represented by —S—R₃, wherein R₃ represents a substituted alkyl or substituted aryl group. The group represented by X⁻ may comprise essentially any negatively charged substituent. Furthermore, a substituted sulfonimide group could be employed in some applications, in association with an R₄ group. Furthermore, it is possible to include CF₂CF₂OCF═CF₂ or CF₂CF₂OCF(CF₃)CF₂OCF═CF₂. Furthermore, as shown in FIG. 2B the R₅ group in that structure may be composed of any number of moities, including but not limited to a halogen atom (such as Fluorine), an alkyl group with one or more substituents, a cycloalkyl group, which may have one or more substituents, or a aryl group which may or may not have one or more substituents.

In the invention, a zwitterionic compound having the structure shown in FIG. 2A may be employed. In the compound, the X⁻ is selected from the group of compounds consisting of: sulphur-containing groups, nitrogen-containing groups and fluorine-containing groups. Furthermore, the compound as employed in FIG. 2A also may include an X⁻ group, in some applications of the invention, in which the X⁻ comprises one or more of the following:

—SO_{3,} —SO₂CHSO₂—R_{5,} —SO₂C (SO₂R₅)₂,

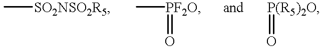

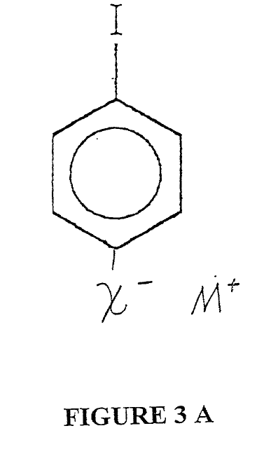

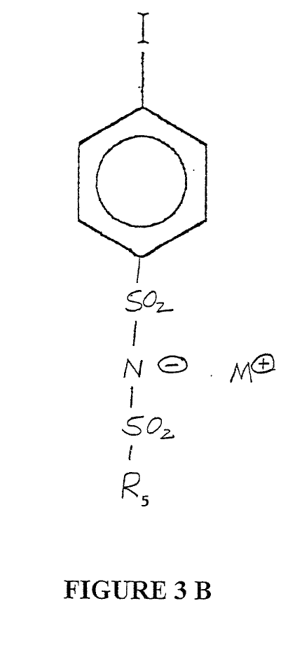

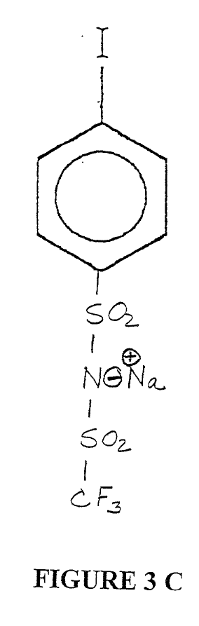

A preferred compound that may be employed in this invention is 4-I—C₆H₄—SO₂NHSO₂R (as in FIG. 3B), which may be reacted to produce an iodonium salt, Ph-I(+)—C₆H₄-4-SO₂N(−)SO₂R₅. As shown in FIG. 2B, the R₅ group may be comprised of any number of moities, including but not limited to: substituted or unsubstituted, hydroxyl alkyls, aryls, cycloalkyls, fluorinated alkyls, and fluorinated cycloalkyls. One preferred zwitterionic compound that may be employed in the practice of this invention is iodoaromatic sulfonimide, 4-I—C₆H₄—SO₂NHSO₂CF₃, which may be prepared and converted into a zwitterionic iodonium salt, Ph-I(+)—C₆H₄-4-SO₂N(−)SO₂CF₃. In this specific embodiment of the invention, the fluorinated carbon atom is the R₅ group of FIG. 2B, and this specific compound may be seen in FIG. 2C.

The group that comprises the fluorinated carbon atom in FIG. 2C may be comprised of other entities instead. This zwitterionic molecule surprisingly has been found to be capable of acting as a photacid generator. The use of this zwitterion provides a photoresist system that is significantly improved over previously known systems. The compositions of this invention are capable of successfully achieving the etch of features on the surface of the circuit with a line width of less than about 0.5 μm lithography.

The choice of a synthetic route for iodonium compounds must consider whether the desired product is symmetrical (i.e. the two aromatic rings are the same) or not symmetrical. Further, it must be considered if the aromatic rings bear electron-withdrawing or electron-donating substituents, and how best to introduce the desired counterion.

Synthesis of these compounds may involve preparing the simplest structure, i.e. a symmetrical salt with no substituents on the rings, such as diphenyl iodonium iodide. There are in principle at least two approaches for such synthesis: (1) iodine is oxidized to a species that can react with two molecules of benzene, or (2) iodobenzene is oxidized to a trivalent iodine intermediate, which then binds to benzene.

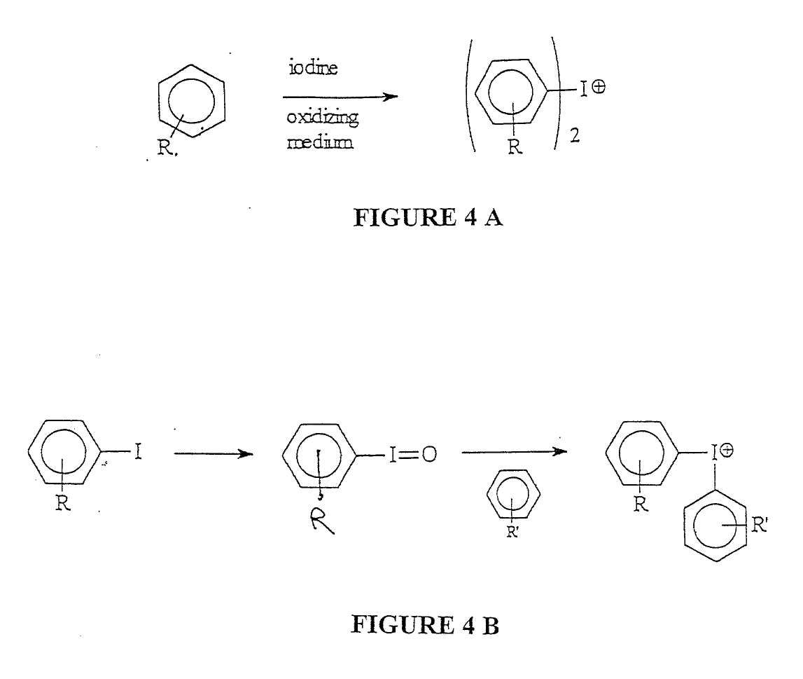

First, and in the case of symmetrical diaryl iodonium zwitterionic salt structures with two rings (i.e.: A₁ arene and A₂ arene, respectively) have a benzene or a substituted benzene. The nature of the substituents will determine the choice of reaction conditions, as will be recognized by a person of skill in the art. FIG. 4A shows the reaction to produce a symmetrical diaryl iodonium salt.

In general, when iodine and two equivalents of an aromatic compound are used, electron-donating substituents do not tolerate strongly oxidizing media. Further, electron-withdrawing substituents may retard the reaction.

The second pathway employs an iodoarene to prepare unsymmetrical diaryl iodonium salts (see FIG. 4B). When an iodoarene is oxidized first, its oxidation is favored by electron-donating substituents. The further reaction with another arene generally follows the synthesis protocol of electrophilic reactions. Electron donating substituents favor the reaction and tend to be para-directing. Electron withdrawing substituents react slowly and generally are meta-directing.

Representative data of systematic investigations are tabulated below, although this list is by no means the exclusive list of all possibilities that may be synthesized pursuant to this invention. This list represents merely examples to illustrate the numerous combinations possible in the synthesis of such compounds for use in this invention.

Symmetrical salts made using iodine are presented first, then symmetrical salts made from an iodoarene, and then finally unsymmetrical salts are presented last. Methods A, B, C, D are explained following Table 1. The first arene, A₁, and the second arene, A₂, are shown in column 1 and column 2, respectively, of Table 1.

TABLE 1
Representative Preparations of Iodonium Salts.

| 1^{st} Arene | 2^{nd} Arene | | Major | Isolated | Yield |
|---|---|---|---|---|---|
| (A₁) | (A₂) | Method | isomer | as X⁻= | % |
| Benzene | Benzene | A | — | Br | 72 |
| Fluorobenzene | Fluorobenzene | A | para | Br | 65 |
| Nitrobenzene | Nitrobenzene | A | meta | Br | 42 |
| Benzoic acid | Benzoic acid | A | meta | I | 53 |
| Toluene | Toluene | B | para | I | 64 |
| Anisole | Anisole | B | para | I | 9 |
| Toluene | Toluene | C | para | CF₃CO₂ | 49 |
| Anisole | Anisole | C | para | Br | 58 |
| Iodobenzene | Benzene | D | — | Br | 74 |
| 2-Nitroiodobenzene | Benzene | D | — | Br | 10 |
| 3-Nitroiodobenzene | Benzene | D | — | Br | 98 |
| 4-Nitroiodobenzene | Benzene | D | — | Br | 41 |
| 2-Iodobenzoic acid | Benzene | D | — | I | 39 |
| 3-Iodobenzoic acid | Benzene | D | — | I | 55 |
| 4-Iodobenzoic acid | Benzene | D | — | I | 69 |
| 4-Fluoroiodo- | Benzene | D | — | Br | 27 |
| benzene | | | | | |
| 4-Iodoanisole | Benzene | D | — | CF₃CO₂ | 92 |
| 3-Acetamidoiodobz. | Benzene | D | — | I | 40 |
| 4-Acetamidoiodobz. | Benzene | D | — | I | 20 |

Method A: Iodine + oxidizer + aromatic in sulfuric acid.
Example: I₂ + HNO₃/H₂SO₄ + PhNO₂ → [(O₂NC₆H₄)₂I]⁺HSO₄⁻
Method B: Iodate + aromatic in acetic anhydride/sulfuric acid.
Example: KIO₃ + benzene + Ac₂O/H₂SO₄ → [(C₆H₅)₂I]⁺HSO₄⁻
Method C: I(OCOCF₃)₂ + aromatic in acetic anhydride.
Example: anisole + I(OCOCF₃)₂/Ac₂O → [(4-CH₃O—C₆H₄)₂I]⁺OCOCF₃⁻
Method D: ArI═O or ArI(OCOCH₃)₂ or (ArI + oxidizer) + other aromatic + acid.
Example: 4-HO₂C—C₆H₄—I + K₂S₂O₈/H₂SO₄ + benzene → → [(4-HO₂C—C₆H₄—I)₂]I⁺HSO₄⁻

Iodonium salts shown above are cation/anion pairs, where the anion comes from the reaction system or is introduced after the reaction by metathesis.

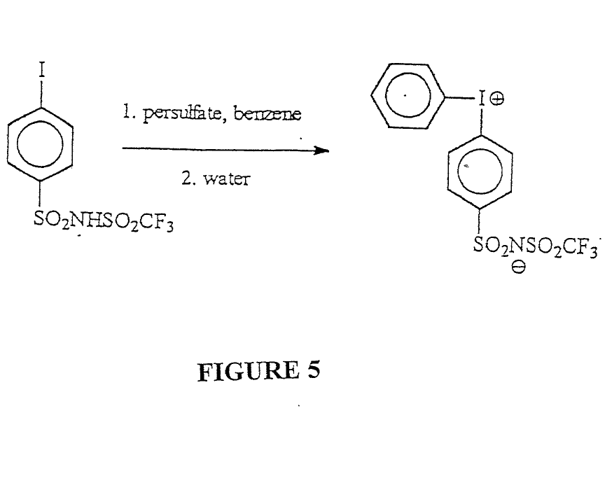

For example, the preparation of Ph-I(+)—C₆H₄-4-SO₂N(−)SO₂CF₃ (see FIG. 5) follows the procedure developed for an unsymmetrical iodonium salt as illustrated in FIG. 4B, where the starting iodoarene bears an electron-withdrawing substituent (see Table 1). The reaction proceeds in the presence of perfulfate, benzene, and water. A similar procedure of synthesis from an iodobenzenesulfonic acid may also be used. Further, other groups may be attached to the iodoarene in place of the SO₂N(−)SO₂CF₃, and the synthesis could occur in the same manner. The group may be an SO₂—N—SO₂R₄ group in which R₄ is selected from an alkyl or fluorocarbon, as examples.

The sulfonimide functional group has the surprisingly unexpected property of allowing the reaction of FIG. 5 to proceed directly as shown in the Figure.

Procedures for the preparation of a sulfonimide-based iodonium zwitterions are provided below. The compound 4-I—C₆H₄—SO₂NHSO₂CF₃ may be reacted with benzene, fluorobenzene, toluene, phenylacetic acid and benzotrifluoride.

Irradiation of 4-F—C₆H₄—I—C₆H₄—SO₂NHSO₂CF₃dissolved in DMSO (dimethylsulfoxide) gave in 20 min substantial decomposition as observed by proton and fluorine Nuclear Magnetic Resonance. DMSO was used because it is the best solvent for the salt. However, it absorbs UV below 300 nm; solvents that let UV pass down to 200 nm also could be used (i.e. (CH₃CN, CF₃CH₂OH, (CF₃)₂CHOH)). TGA (Thermal Gravimetric Analysis) is a useful analytical technique, which is known by persons of skill in the art.

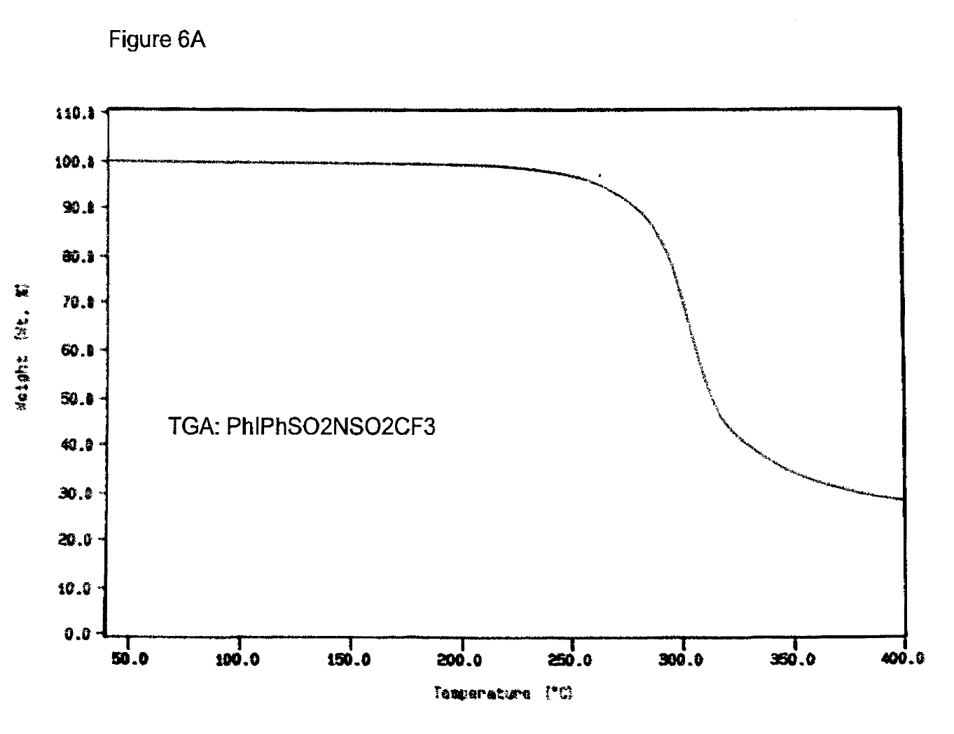

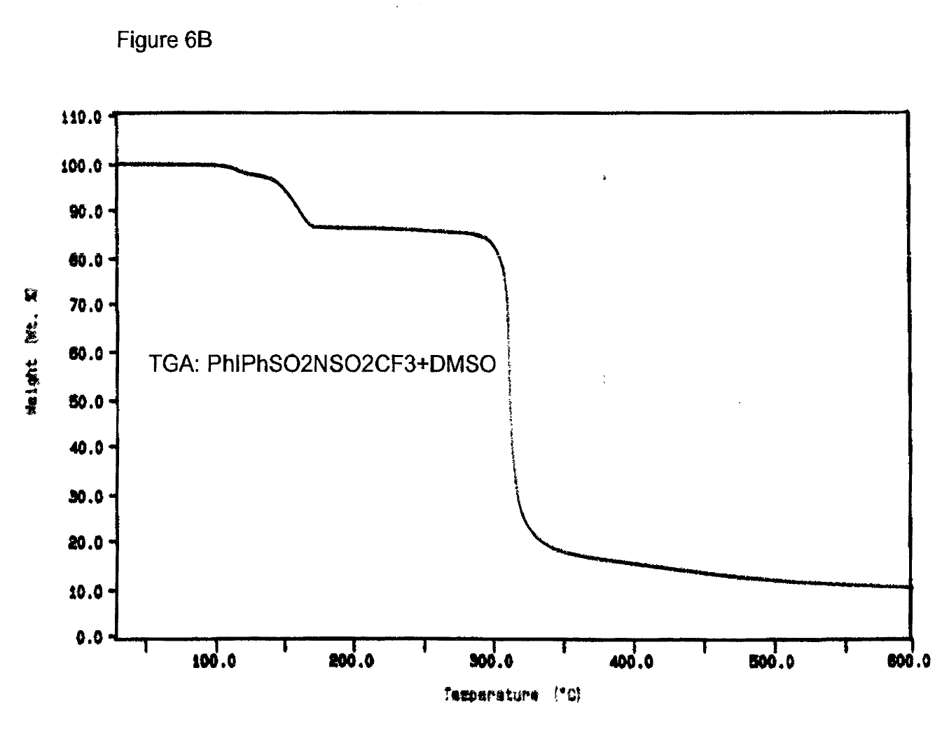

In FIGS. 6A and 6B one can see a TGA analysis for C₆H₅—I—C₆H₄—SO₂NSO₂CF₃, illustrating thermal stability of the Iodonium salt to nearly 300° C.

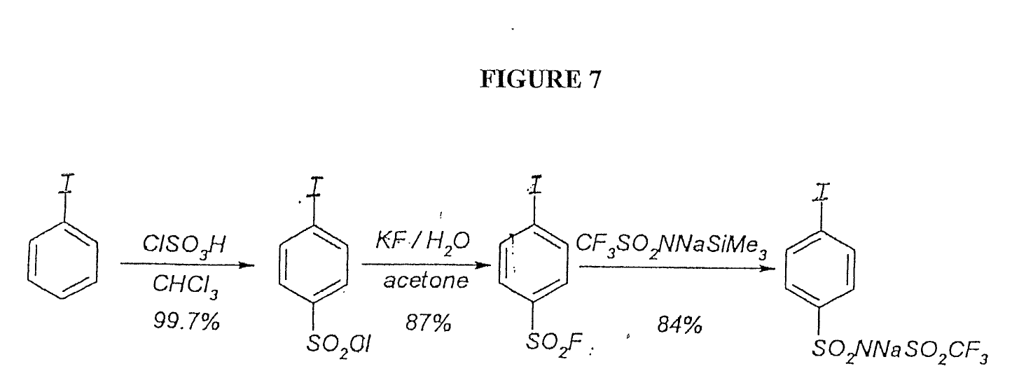

The synthesis of the starting material used in the iodonium salts of this invention proceed in high yield from relatively inexpensive starting materials. The reaction to make the starting iodobenzenesulfonimide can be prepared as shown in FIG. 7. Pipsyl chloride is prepared using a method similar to that described in U.S. Pat. No. 5,684,192. The fluorination of pipsyl chloride is carried out using a method similar to that described in U.S. Pat. No. 3,560,568. The final step is the coupling reaction uses N-trimethylsilyl-trifluoromethanesulfonamide-Na salt which has been shown to readily undergo reactions with R_{f}SO₂F. Although the iodobenzenesulfonimides have shown encouraging and surprisingly unexpected results when used in iodonium salts, modifications of the pendent group also may be desirable as one aspect of this invention as a means of “tuning” or further adjusting the solubility of these compounds. Various iodobenzenesulfonimides also may be synthesized with varying R groups. Listed below are some representative reactions, along with the resulting products if any.

TABLE 2
Reactions to produce Iodobenzenesulfonimides

| Sulfonyl Halide | Sulfonamide | Method | Product |
|---|---|---|---|
| IC₆H₄SO₂Cl | C₆H₅SO₂NH₂ | A | No rxn |
| IC₆H₄SO₂F | C₆H₅SO₂NH₂ | A | No rxn |
| CF₂BrCFBrOCF₂CF₂SO₂F | IC₆H₄SO₂N | B | CF₂BrCFBrOCF₂ |
| | NaSiMe₃ | | CF₂SO₂NaSO₂C₆ |
| | | | H₄I |
| IC₆H₄SO₂Cl | C₆H₅SO₂NH₂ | C | No rxn |
| IC₆H₄SO₂F | CH₃SO₂NH₂ | C | No rxn |
| IC₆H₄SO₂F | C₆H₅SO₂NH₂ | C | No rxn |
| CIC(O)OCH₂Ph | C₆H₅SO₂NH₂ | C | C₆H₅SO₂NH-Z |

A: RSO₂NH₂ + LiOH → RSO₂NHLi + R′SO₂X → RSO₂NHSO₂R′
B: RSO₂NNaSiMe₃ + R′SO₂X → RSO₂NNaSO₂R′
C: DIEA, CH₃CN + RSO₂NH₂

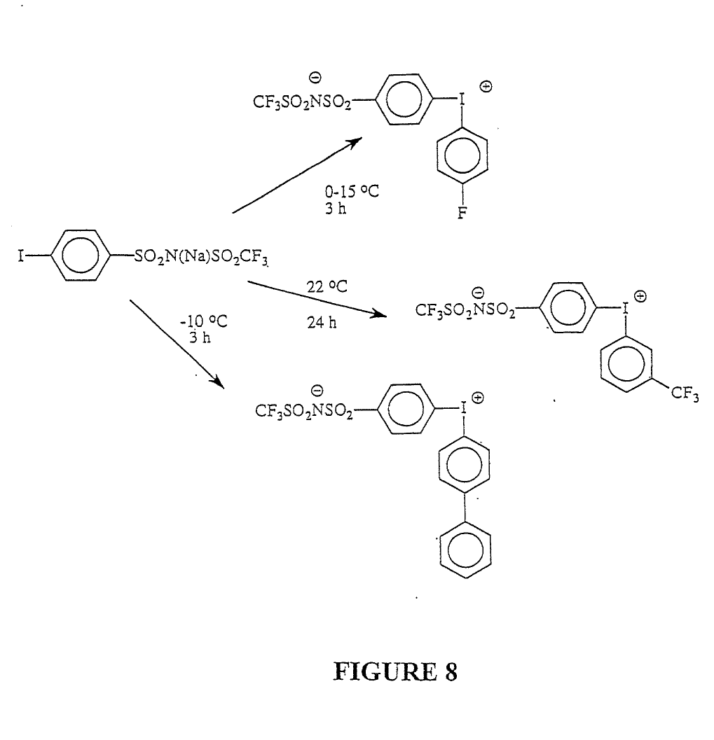

A further method of implementing the present invention is by way of a reaction pathway which serves to increase the reactivity of the sulfonyl halide as well as increase the solubilty of the aromatic system, as further shown in FIG. 8. In FIG. 8, the nitrobenzene sulfonyl chloride undergoes reaction with ArSO₂NHLi to produce the corresponding coupled product (i.e. the di-aryl product). Once the coupled product is formed, a deamination/halogenation reaction can be employed to produce the corresponding iodine compound. This reaction pathway facilitates the exploration of ortho, meta, and para directed products by using different substitued starting materials. As will be recognized by a person of skill in the art, numerous possibilities are available in the practice of the invention, and the products shown in FIG. 8 comprise just three of many possibilities of the zwitterionic compounds that may be produced in the practice of this invention.

It is understood by one of ordinary skill in the art that the present discussion is a description of exemplary embodiments only, and is not intended as limiting the broader aspects of the present invention, which broader aspects are embodied in the exemplary constructions.

1. A zwitterionic compound, comprising: [chemical structure]
wherein X⁻ is selected from the group of compounds consisting of: sulfur-containing groups, nitrogen-containing groups, and fluorine-containing groups; and
wherein R₁ and R₂ are each independently selected from the group consisting of substituted or unsubstituted: alkyls, aryls, halides, and fluorinated alkyls, nitrogen containing groups, halogenated alkyls, alkoxy, aryloxy, halogenated alkoxy, unsaturated alkyls, thioalkyls, unsaturated fluorinated alkyls, unsaturated alkoxy keto alkyls, alkoxys, aryloxy, keto aryls, sulfonyl alkyl, sulfonyl aryls.

2. The compound of claim 1 in which X⁻ comprises one of the following entities selected from the group consisting of:
—SO₃, —SO₂CHSO₂—R₅, —SO₂C (SO₂R₅)₂, 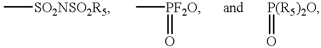
wherein R₅ is selected from the group consisting of substituted or unsubstituted alkyls, aryls, cycloalkyls, fluorinated alkyls, and fluorinated cycloalkyls.

3. The compound of claim 2 in which X⁻ comprises:
—SO₂—N—SO₂—R₅.

4. The compound of claim 2 in which X⁻ comprises:
—SO₂—N—SO₂—CF₃.

5. The compound of claim 3 in which R₅ is a fluorinated alkyl.

6. The compound of claim 5 in which R₅ is a substituted or unsubstituted aryl.

7. A diaryl zwitterionic iodonium salt having an iodine atom that is positively charged and an anionic group which comprises either a first substituted arene group designated A₁ or a second substituted arene group designated A₂, in which A₁ and A₂ are each independently selected from the group consisting of: benzene, fluorobenzene, nitrobenzene, benzoic acid, toluene, anisole, iodobenzene, 2-nitroiodobenzene, 3-nitroiodobenzene, 4-nitroiodobenzene, 2-iodobenzoic acid, 3-iodobenzoic acid, 4-iodobenzoic acid, fluoroiodobenzene 4-iodoanisole, 3-acetamidoiodobenzene, and 4- acetamidoiodobenzene;
further wherein either A₁ or A₂ contain an SO₂—N—SO₂R₅ group substituted upon the arene ring;
wherein R₅ is selected from the group consisting of substituted or unsubstituted alkyls, aryls, cycloalkyls, fluorinated alkyls, and fluorinated cycloalkyls.

8. The salt compound of claim 7 in which the first arene group A₁ is benzene, and the second arene group A₂ is benzene.

9. The salt compound of claim 7 in which the first arene group A₁ is nitrobenzene, and the second arene group A₂ is nitrobenzene.

10. The salt compound of claim 7 in which the first arene group A₁ is benzoic acid, and the second arene group A₂ is benzoic acid.

11. The salt compound of claim 7 in which the first arene group A₁ is toluene, and the second arene group A₂ is toluene.

12. The salt compound of claim 7 in which the first arene group A₁ is anisole, and the second arene group A₂ is anisole.

13. The salt compound of claim 7 in which the first arene group A₁ is Iodobenzene, and the second arene group A₂ is benzene.

14. The salt compound of claim 7 in which the first arene group A₁ is 2-Nitroiodobenzene, and the second arene group A₂ is benzene.

15. The salt compound of claim 7 in which the first arene group A₁ is 3-Nitroiodobenzene, and the second arene group A₂ is benzene.

16. The salt compound of claim 7 in which the first arene group A₁ is 3-Nitroiodobenzene, and the second arene group A₂ is benzene.

17. The salt compound of claim 7 in which the first arene group A₁ is 4-Nitroiodobenzene, and the second arene group A₂ is benzene.

18. The salt compound of claim 7 in which the first arene group A₁ is 2-Iodobenzoic acid, and the second arene group A₂ is benzene.

19. The salt compound of claim 7 in which the first arene group A₁ is 3-Iodobenzoic acid, and the second arene group A₂ is benzene.

20. The salt compound of claim 7 in which the first arene group A₁ is 4-Iodobenzoic acid, and the second arene group A₂ is benzene.

21. A wafer having lithographically etched features on the surface of the wafer, the etched features being adapted for forming an electrical circuit, the etched features having a line width of less than about 0.5μ, whereby the etch is formed in a photoresist process that employs a diaryl zwitterionic iodonium salt.
